(19) United States
(12) Patent Application Publication
(10) Pub. No.: US 2022/0122697 A1
(43) Pub. Date: Apr. 21, 2022

(54) METHOD FOR TRAINING COMPOUND PROPERTY PREDICTION MODEL AND METHOD FOR PREDICTING COMPOUND PROPERTY

(71) Applicant: Beijing Baidu Netcom Science Technology Co., Ltd., Beijing (CN)

(72) Inventors: Lihang LIU, Beijing (CN); Jieqiong Lei, Beijing (CN); Xiaomin Fang, Beijing (CN); Donglong He, Beijing (CN); Fan Wang, Beijing (CN)

(21) Appl. No.: 17/565,282

(22) Filed: Dec. 29, 2021

(30) Foreign Application Priority Data
May 26, 2021 (CN) 202110577762.8

(51) Int. Cl. G16C 20/30; G16C 20/70; G16C 20/20; G06N 5/02

(52) U.S. Cl. CPC G16C 20/30; G06N 5/025; G16C 20/20; G16C 20/70

(57) ABSTRACT

A method for predicting a compound property, apparatuses, an electronic device, a computer readable storage medium, and a computer program product are provided. The method includes: for each first sample compound of first sample compounds, acquiring spatial structure information of a spatial structure formed by atoms and chemical bonds that constitute the first sample compound; training, using the first sample compounds as input samples and pieces of corresponding spatial structure information as output samples, to obtain a spatial structure prediction model; and continuing training, using second sample compounds as input samples and pieces of corresponding property information as output samples, to obtain the compound property prediction model on the basis of the spatial structure prediction model.

### CROSS-REFERENCE TO RELATED APPLICATIONS

The present application claims the priority of Chinese Patent Application No. 202110577762.8, titled “METHOD FOR TRAINING COMPOUND PROPERTY PREDICTION MODEL AND METHOD FOR PREDICTING COMPOUND PROPERTY”, filed on May 26, 2021, the content of which is incorporated herein by reference in its entirety.

### TECHNICAL FIELD

The present disclosure relates to a field of artificial intelligence, in particular to a field of deep learning and neural network technology, and more particular to a method for training a compound property prediction model and a method for predicting a compound property, as well as corresponding apparatuses, an electronic device, a computer readable storage medium, and a computer program product.

### BACKGROUND

In recent years, drug design driven by AI (Artificial Intelligence) has received more attention than traditional biological experiments. Therefore, using deep learning methods to facilitate accurate prediction of drug molecules has become more and more important, for example, drug toxicity prediction, affinity prediction of drug ligands and protein receptors, etc.

### SUMMARY

Embodiments of the present disclosure propose a method for training a compound property prediction model and a method for predicting a compound property, apparatuses, an electronic device, a computer readable storage medium, and a computer program product.

In a first aspect, a method for training a compound property prediction model is provided in some embodiments of the present disclosure, including: for each first sample compound of first sample compounds, acquiring spatial structure information of a spatial structure formed by atoms and chemical bonds that constitute the first sample compound; training, using the first sample compounds as input samples and pieces of corresponding spatial structure information as output samples, to obtain a spatial structure prediction model; and continuing training, using second sample compounds as input samples and pieces of corresponding property information as output samples, to obtain the compound property prediction model on the basis of the spatial structure prediction model, wherein an order of magnitudes of the second sample compounds labeled with the pieces of corresponding property information being less than an order of magnitudes of the first sample compounds that are not labeled with corresponding property information.

In a second aspect, an apparatus for training a compound property prediction model is provided in some embodiments of the present disclosure, including: a spatial structure information acquisition unit, configured to, for each first sample compound of first sample compounds, acquire spatial structure information of a spatial structure formed by atoms and chemical bonds that constitute the first sample compound; a spatial structure prediction model training unit, configured to train, using the first sample compounds as input samples and pieces of corresponding spatial structure information as output samples, to obtain a spatial structure prediction model; and a compound property prediction model training unit, configured to continue training, using second sample compounds as input samples and pieces of corresponding property information as output samples, to obtain the compound property prediction model on the basis of the spatial structure prediction model, wherein an order of magnitudes of the second sample compounds labeled with the pieces of corresponding property information being less than an order of magnitudes of the first sample compounds that are not labeled with corresponding property information.

In a third aspect, some embodiments of the present disclosure provide a non-transitory computer-readable medium storing a computer program thereon, where the program, when executed by a processor, implements the method for training a compound property prediction model as described in the first aspect.

It should be understood that the content described in this section is not intended to identify key or important features of the embodiments of the present disclosure, nor is it intended to limit the scope of the present disclosure. Other features of the present disclosure will be easily understood through the following specification.

### BRIEF DESCRIPTION OF THE DRAWINGS

By reading the detailed description of non-limiting embodiments with reference to the following accompanying drawings, other features, objects and advantages of the present disclosure will become more apparent:

FIG. 1 is an exemplary system architecture to which the present disclosure may be applied;

FIG. 2 is a flowchart of a method for training a compound property prediction model according to an embodiment of the present disclosure;

FIG. 3 is a flowchart of a method for acquiring spatial structure information of a sample compound according to an embodiment of the present disclosure;

FIG. 4 is a flowchart of another method for training a compound property prediction model according to an embodiment of the present disclosure;

FIG. 5 is a structural block diagram of an apparatus for training a compound property prediction model according to an embodiment of the present disclosure;

FIG. 6 is a structural block diagram of an apparatus for predicting a compound property according to an embodiment of the present disclosure; and

FIG. 7 is a schematic structural diagram of an electronic device suitable for executing the method for training a compound property prediction model and/or the method for predicting a compound property according to an embodiment of the present disclosure.

### DETAILED DESCRIPTION OF EMBODIMENTS

The following describes exemplary embodiments of the present disclosure in conjunction with the accompanying drawings, which includes various details of the embodiments of the present disclosure to facilitate understanding, and they should be considered as merely exemplary. Therefore, those of ordinary skill in the art should recognize that various changes and modifications may be made to the embodiments described herein without departing from the scope and spirit of the present disclosure. Also, for clarity and conciseness, descriptions of well-known functions and structures are omitted in the following description. It should be noted that embodiments in the present disclosure and the features in embodiments may be combined with each other on a non-conflict basis.

In the technical solution of the present disclosure, the acquisition, storage, and application of user personal information involved are in compliance with relevant laws and regulations, and necessary confidentiality measures have been taken, and they do not violate public order and good customs.

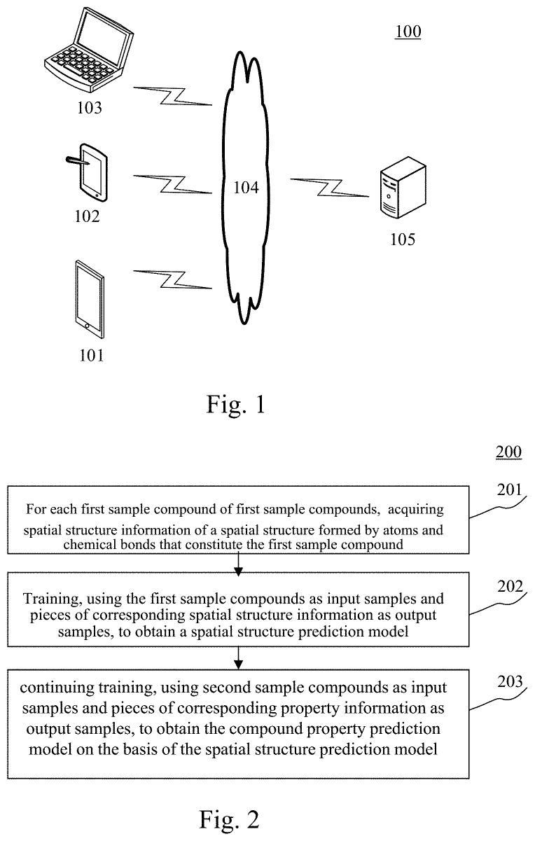

FIG. 1 shows an exemplary system architecture 100 to which embodiments of a method for training a face recognition model, a method for training a compound property prediction model, apparatuses, an electronic device, and a computer readable storage medium of the present disclosure may be applied.

As shown in FIG. 1, the system architecture 100 may include terminal devices 101, 102, 103, a network 104 and a server 105. The network 104 serves as a medium for providing a communication link between the terminal devices 101, 102, 103 and the server 105. The network 104 may include various types of connections, such as wired or wireless communication links, or optical fiber cables.

A user may use the terminal devices 101, 102, 103 to interact with the server 105 through the network 104 to receive or send messages, and so on. The terminal devices 101, 102, 103 and the server 105 may be installed with various applications for implementing information communication between the two, such as molecular dynamics simulation applications, model training applications, or model calling applications.

The terminal devices 101, 102, 103 and the server 105 may be hardware or software. When the terminal devices 101, 102, 103 are hardware, they may be various electronic devices having display screens, including but not limited to smart phones, tablet computers, laptop computers, desktop computers, etc.; when the terminal devices 101, 102, 103 are software, they may be installed in the electronic devices listed above. They may be implemented as a plurality of software or software modules, or as a single software or software module, which is not limited herein. When the server 105 is hardware, it may be implemented as a distributed server cluster composed of a plurality of servers, or as a single server; when the server is software, it may be implemented as a plurality of software or software modules, or as a single software or software module, which is not limited herein.

The server 105 may provide various services using various built-in applications. Taking a model calling application that can provide users with compound property prediction services as an example, the server 105 may achieve the following effects when running the model calling application: firstly, acquiring a to-be-determined compound with properties to be determined transmitted from the terminal devices 101, 102, 103 through the network 104; then, calling a preset compound property prediction model stored in a preset position to predict property information of the to-be-determined compound.

The compound property prediction model may be obtained through training by a built-in model training application on the server 105 according to the following steps: firstly, for each first sample compound of first sample compounds, acquiring spatial structure information of a spatial structure formed by atoms and chemical bonds that constitute a first sample compound; then training, using the first sample compounds as input samples and pieces of corresponding spatial structure information as output samples, to obtain a spatial structure prediction model; and continue training, using second sample compounds as input samples and pieces of corresponding property information as output samples, on the basis of the spatial structure prediction model, to obtain the compound property prediction model. An order of magnitudes of the second sample compounds labeled with the pieces of corresponding property information being less than an order of magnitudes of the first sample compounds that are not labeled with corresponding property information.

Since training to obtain the compound property prediction model requires a lot of computing resources and strong computing power, the method for training a compound property prediction model provided in the subsequent embodiments of the present disclosure is generally executed by the server 105 having strong computing power and more computing resources. Correspondingly, the apparatus for training a compound property prediction model is generally provided in the server 105. But at the same time, it should also be noted that when the terminal devices 101, 102, 103 also have the computing power and computing resources that meet the requirements, the terminal devices 101, 102, 103 may also use compound property prediction model training applications installed thereon to complete the above calculations that were originally assigned to the server 105 to output the same results as the server 105. Correspondingly, the apparatus for training a compound property prediction model may also be provided in the terminal devices 101, 102, 103. In this case, the exemplary system architecture 100 may not include the server 105 and the network 104.

Of course, the server used to train to obtain the compound property prediction model may be different from a server that calls the trained compound property prediction model to use. In particular, the compound property prediction model trained by the server 105 may also be used to obtain a lightweight compound property prediction model suitable for being placed in the terminal devices 101, 102, 103 through model distillation, that is, it may flexibly choose to use the lightweight compound property prediction model in the terminal devices 101, 102, 103 or the more complex compound property prediction model in the server 105 according to a recognition accuracy of actual needs.

It should be appreciated that the number of the terminal devices, the network and the server in FIG. 1 is merely illustrative. Any number of terminal devices, networks and servers may be provided according to actual requirements.

With reference to FIG. 2, FIG. 2 is a flowchart of a method for training a compound property prediction model according to an embodiment of the present disclosure, where a flow 200 includes the following steps.

Step 201: for each first sample compound of first sample compounds, acquiring spatial structure information of a spatial structure formed by atoms and chemical bonds that constitute the first sample compound;

This step aims to acquire the spatial structure information of the first sample compound by an executing body of the method for training a compound property prediction model (for example, the server 105 shown in FIG. 1).

Different from a simple substance composed of only one kind of atom, a compound is composed of at least two kinds of different atoms, and various chemical bonds are formed between the atoms. Therefore, the spatial structure information mainly relates to a spatial structure formed by atoms and chemical bonds, such as bond angles and bond lengths of the chemical bonds, three-dimensional coordinates of respective atoms, an overall potential energy of compound molecule, atomic distances, and so on. Specifically, the several types of spatial structure information mentioned above may be determined through molecular dynamics simulation applications or related experiments.

It should be noted that, since the spatial structure is formed based on a basic planar structure with further increased dimensions, the spatial structure information described in the present disclosure actually also includes basic planar structure information.

A reason for acquiring the spatial structure information is that from a microscopic point of view, downstream tasks such as a property prediction of compound molecules and an interaction between a drug and a target are essentially results of intermolecular interactions (proteins may be regarded as macromolecules), and this process is closely related to the spatial structure and energy of a molecule. Therefore, the acquisition of the spatial structure information is a basis for identifying the interaction.

Step 202: training, using the first sample compounds as input samples and pieces of corresponding spatial structure information as output samples, to obtain a spatial structure prediction model;

On the basis of step 201, this step aims to perform, by the executing body, training to obtain the spatial structure prediction model which learns, from a sample pair of a first sample compound used as an input sample and a piece of corresponding spatial structure information used as an output sample, a correspondence between the sample pair. Taking the overall potential energy as an example, the spatial structure prediction model may be an overall potential energy prediction model, that is, a trained overall potential energy prediction model can represent a correspondence between a compound and the overall potential energy of the compound.

It should be understood that it is relatively easy to acquire the spatial structure information of a compound (as opposed to acquiring property information of the compound) by means of simulation tools such as molecular dynamics simulation or means such as experimental calculation. Therefore, the training sample pair used in this step has a relatively large order of magnitudes, and it is intended that the spatial structure prediction model trained based on this can learn relevant knowledge to identify the spatial structure of the compound.

That is, the spatial structure prediction model starts from an initialized blank model, and is trained using the first sample compounds as the input samples and the piece of corresponding spatial structure information as the output samples.

Step 203: continuing training, using second sample compounds as input samples and pieces of corresponding property information as output samples, to obtain the compound property prediction model on the basis of the spatial structure prediction model.

In this step, on the basis of the spatial structure prediction model trained in step 202, the executing body may continue training, to obtain the compound property prediction model which learns a correspondence from a sample pair of a second sample compound used as an input sample and a piece of corresponding property information used as an output sample.

That is, different from a training process of the spatial structure prediction model, the compound property prediction model no longer uses an initialized blank model as a training basis, but directly uses a previously trained spatial structure prediction model as the training basis, and then uses the second sample compound as the input sample and the corresponding property information as the output sample and is obtained through training.

Since being based on the spatial structure prediction model that can represent the correspondence between a compound and the overall potential energy of the compound, the compound property prediction model trained in this step can also represent the correspondence between the spatial structure and the properties of a compound. The reason is that the properties of a compound are inherently related to its spatial structure.

The property information may include at least one of water solubility, toxicity, a matching degree with preset protein, compound reaction characteristics, stability, or degradability. Of course, in addition to several compound properties listed above, there may also be other different properties exhibited due to different spatial structures of the compound, which will not be listed herein.

Here, an order of magnitudes of the second sample compounds labeled with the pieces of property information is less than an order of magnitudes of the first sample compounds that are not labeled with corresponding property information, and a difference in the order of magnitudes is usually from 10³ to 10⁴. Based on an actual quantity of the second sample compounds labeled with the pieces of corresponding property information, the first sample compounds not labeled with corresponding property information and with an order of magnitudes of at least 10³ to 10⁴ higher than the second sample compound are selected. For example, when a total number of the second sample compounds labeled with the pieces of corresponding property information is several thousand, it is generally required that a total number of the first sample compounds not labeled with corresponding property information has an order of magnitudes of 100,000 to tens of millions, so that when the total number of the second sample compound is small, a compound property prediction model having a high accuracy can be obtained by training.

In the method for training a compound property prediction model provided by the embodiments of the present disclosure, by means of the first sample compounds with a large sample quantity and spatial structure information thereof, firstly the spatial structure prediction model from which relevant knowledge of the spatial structure information is learnt. Then, on the basis of the spatial structure prediction model with relevant knowledge of the spatial structure information, the second sample compounds labeled with the pieces of corresponding property information and with a smaller sample quantity are used to continue training, that is, the direct correspondence between the original spatial structure and the properties is split into two parts for sequential training, making full use of a large amount of sample compound data that is not labeled with property information. As such, when the number of sample compounds labeled with corresponding property information is small, a compound property prediction model having a high prediction accuracy is obtained.

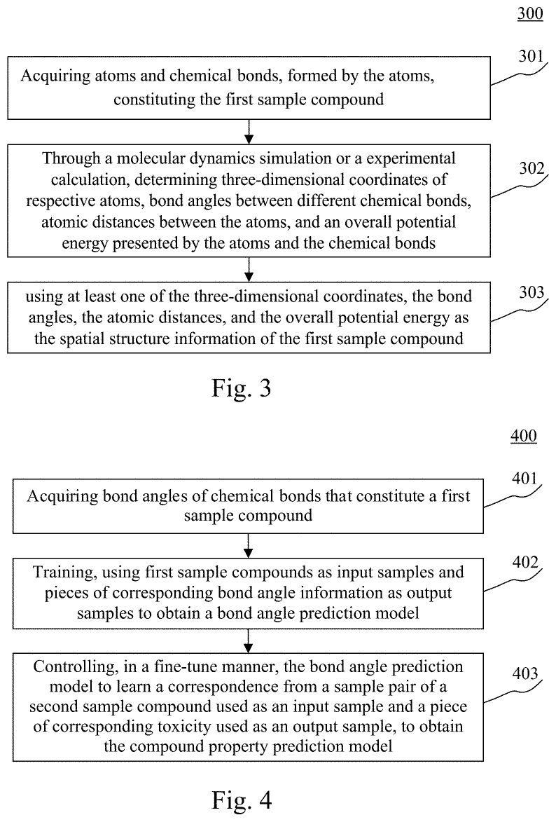

With further reference to FIG. 3, FIG. 3 is a flowchart of a method for acquiring spatial structure information of a sample compound according to an embodiment of the present disclosure. That is, an implementation is provided for step 201 in the flow 200 shown in FIG. 2, and other steps in the flow 200 are not adjusted. The implementation provided in the present embodiment is also used to replace step 201 to obtain a new and complete embodiment. The flow 300 includes the following steps.

Step 301: acquiring the atoms and the chemical bonds, formed by the atoms, constituting the first sample compound;

Step 302: through a molecular dynamics simulation or a experimental calculation, determining three-dimensional coordinates of respective atoms, bond angles between different chemical bonds, atomic distances between the atoms, and an overall potential energy presented by the atoms and the chemical bonds;

On the basis of step 301, this step aims to acquire different spatial structure information describing the spatial structure of the compound from different perspectives by the executing body through molecular dynamics simulation or experimental calculation.

Molecular dynamics simulation is a simulation tool that may simulate a specific structure of a molecule in a virtual space based on preset database information, and determine a possible spatial structure based on a preset structural stability criterion.

Step 303: using at least one of the three-dimensional coordinates, the bond angles, the atomic distances, and the overall potential energy as the spatial structure information of the first sample compound.

On the basis of step 302, this step aims to use at least one of the three-dimensional coordinates, the bond angles, the atomic distances, and the overall potential energy as the spatial structure information of the first sample compound by the executing body.

Based on compound properties nowadays, the bond angle between the chemical bonds is an important factor that leads to the formation of the spatial structure of the molecules that constitute the compound. Therefore, in scenarios where a high accuracy is not required, only the bond angle between the chemical bonds may be used as unique spatial structure information. For scenarios having high accuracy requirements, the bond angle between the chemical bonds may also be used as core spatial structure information, and the three-dimensional coordinates, the atomic distances, and the overall potential energy and the like may be used as auxiliary supplementary spatial structure information to improve the accuracy of discrimination as much as possible by integrating the core spatial structure information and the auxiliary supplementary spatial structure information.

On the basis of any of the foregoing embodiments, a high-order spatial structure prediction model may also be obtained by superimposing a trained single-layer spatial structure prediction model, so as to meet a possible predictive demand for a correlation between properties corresponding to more complex spatial structures.

Specifically, a first-layer spatial structure prediction model may model features and spatial structures of first-order neighbors, and a second-layer spatial structure prediction model may model features and spatial structures of second-order neighbors, and so on. When superimposing is performed to obtain an n-layer spatial structure prediction model, features and spatial structures of n-order neighbors may be modeled. Therefore, by setting an appropriate n, a high-order or even a complete 3D spatial structure may be modeled, and rich and complex spatial structure information may be directly integrated into a network. In this way, all the features and spatial structures of compound molecules may be taken into consideration, and more comprehensive information may be learnt, thereby improving the performance of the model on various prediction tasks. For example, the tasks are: determining molecular toxicity, accurately identifying targeted drugs through DTI (Drug-Target Interaction), and predicting drug combinations through DDI (Drug-Drug Interaction), etc.

Furthermore, when a complexity of the spatial structure prediction model exceeds a preset complexity, a lightweight spatial structure prediction model may also be obtained through model distillation technology. That is, the model distillation technology may be used to minimize the complexity, the order of magnitudes, and a size of a distilled student model while retaining the prediction accuracy of the complex model (i.e., teacher model) as much as possible.

With reference to FIG. 4, FIG. 4 is a flowchart of another method for training a compound property prediction model according to an embodiment of the present disclosure. Taking the bonding angles of chemical bonds as spatial structure information and the compound toxicity as property information of the compound as an example, a flow 400 includes the following steps:

Step 401: acquiring bond angles of chemical bonds that constitute a first sample compound;

Step 402: training, using first sample compounds as input samples and pieces of corresponding bond angle information as output samples to obtain a bond angle prediction model;

That is, the bond angle prediction model starts from an initialized blank model, and is trained using the first sample compounds as the input samples and the pieces of corresponding bond angle information as the output samples.

Step 403: controlling, in a fine-tune manner, the bond angle prediction model to learn a correspondence from a sample pair of a second sample compound used as an input sample and a piece of corresponding toxicity used as an output sample, to obtain the compound property prediction model.

The fine-tune technology has a full English name of Fine Tune and a technical principle thereof may be generally summarized as follows: firstly learning a structural diagram of a network, and then modifying a part of the network to a model needed. By means of fine-tune, it is possible to start from a pre-trained model and apply the neural network to a data set of one's own.

The compound property prediction model is obtained through training by using the bond angle prediction model as a training basis, using the second sample compound as the input sample and the corresponding toxicity information as the output sample.

In the foregoing embodiments, how to train to obtain the compound property prediction model is described from various aspects. In order to highlight the effect of the trained compound property prediction model from an actual use scenario as much as possible, the present disclosure also provides a solution to actual problems using a trained compound property prediction model, and a method for predicting a compound property includes the following steps:

acquiring a to-be-determined compound with properties to be determined; and

calling a preset compound property prediction model to predict property information of the to-be-determined compound.

An executing body of the present embodiment may be different from the executing body used for training to obtain the compound property prediction model, or may be the same executing body, which may be flexibly selected according to actual needs, and is not limited herein.

In other words, in the model training phase, the technical solution provided by the present disclosure firstly uses large-scale compound molecules that are not labeled with corresponding property information to perform pre-training to learn spatial structure-related knowledge, then uses a trained spatial structure prediction model as the basis, and uses a small sample quantity of compound molecules labeled with pieces of corresponding property information for fine-tuning. This may simplify research and development costs, may directly and effectively train an applicable model without hundreds of millions of parameters and expensive graphics computing resources, and may also improve the property prediction performance of compound and provide users with a better learning experience. Furthermore, the technical solution provided by the present disclosure also develops the richness of spatial structure information from a microscopic perspective to a certain extent, improves the efficiency of drug research and development, and provides an important solution for subsequent solution of challenging pharmaceutical problems.

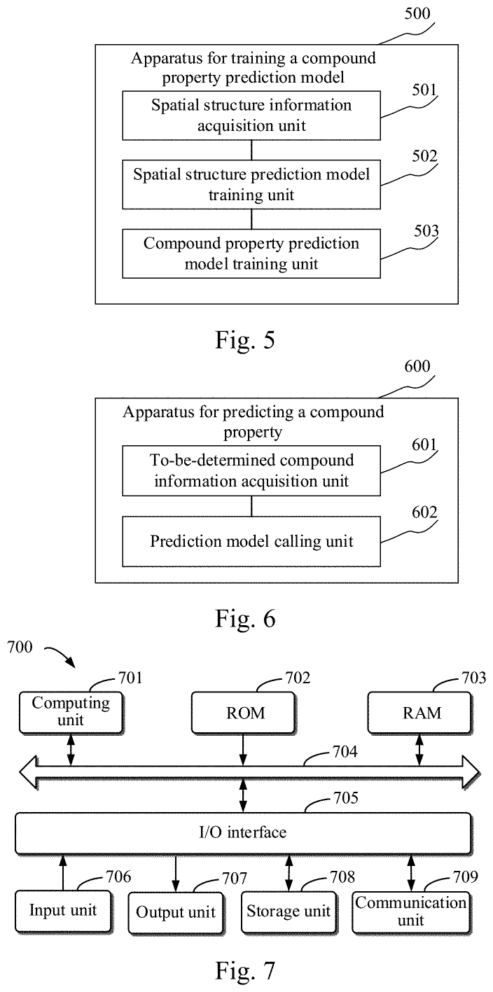

With further reference to FIG. 5 and FIG. 6, as an implementation of the methods shown in the above figures, the present disclosure provides an embodiment of an apparatus for training a compound property prediction model and an embodiment of an apparatus for predicting a compound property. The embodiment of the apparatus for training a compound property prediction model corresponds to the embodiment of the method for training a compound property prediction model as shown in FIG. 2, and the embodiment of the apparatus for predicting a compound property corresponds to the embodiment of the method for predicting a compound property. The apparatuses may be applied to various electronic devices.

As shown in FIG. 5, an apparatus 500 for training a compound property prediction model of the present embodiment may include: a spatial structure information acquisition unit 501, a spatial structure prediction model training unit 502 and a compound property prediction model training unit 503. The spatial structure information acquisition unit 501 is configured, for each first sample compound of first sample compounds, acquire spatial structure information of a spatial structure formed by atoms and chemical bonds that constitute the first sample compound. The spatial structure prediction model training unit 502 is configured to train, using the first sample compounds as input samples and pieces of corresponding spatial structure information as output samples, to obtain a spatial structure prediction model. The compound property prediction model training unit 503 is configured to continue training, using second sample compounds as input samples and pieces of corresponding property information as output samples, to obtain the compound property prediction model on the basis of the spatial structure prediction model, an order of magnitudes of the second sample compounds labeled with the pieces of corresponding property information being less than an order of magnitudes of the first sample compounds that are not labeled with corresponding property information.

In the present embodiment, in the apparatus 500 for training a compound property prediction model: for the specific processing and the technical effects of the spatial structure information acquisition unit 501, the spatial structure prediction model training unit 502 and the compound property prediction model training unit 503, reference may be made to the relevant descriptions of steps 201-203 in the embodiment corresponding to FIG. 2 respectively, and detailed description thereof will be omitted.

In some optional implementations of the present embodiment, the spatial structure information acquisition unit 501 may be further configured to:

acquire the atoms and the chemical bonds, formed by the atoms, constituting the first sample compound;

through a molecular dynamics simulation or a experimental calculation, determine three-dimensional coordinates of respective atoms, bond angles between different chemical bonds, atomic distances between the atoms, and an overall potential energy presented by the atoms and the chemical bonds; and

use at least one of the three-dimensional coordinates, the bond angles, the atomic distances, and the overall potential energy as the spatial structure information of the first sample compound.

In some optional implementations of the present embodiment, the property information of a compound includes at least one of water solubility, toxicity, a matching degree with preset protein, compound reaction characteristics, stability, or degradability.

In some optional implementations of the present embodiment, the compound property prediction model training unit 503 may be further configured to:

control, in a fine-tune manner, the spatial structure prediction model to learn a correspondence from a sample pair of a second sample compound used as an input sample and a piece of corresponding property information used an the output sample, to obtain the compound property prediction model.

In some optional implementations of the present embodiment, the apparatus 500 for training a compound property prediction model may further include:

a model distillation unit, configured to distillate, in response to a complexity of the spatial structure prediction model exceeding a preset complexity, to obtain a lightweight spatial structure prediction model through a model distillation technology.

As shown in FIG. 6, an apparatus 600 for predicting a compound property of the present embodiment may include: a to-be-determined compound information acquisition unit 601 and a prediction model calling unit 602. The to-be-determined compound information acquisition unit 601 is configured to acquire a to-be-determined compound with properties to be determined. The prediction model calling unit 602 is configured to call a preset compound property prediction model to predict property information of the to-be-determined compound, where the compound property prediction model is obtained according to the apparatus 500 for training a compound property prediction model.

In the present embodiment, in the apparatus 600 for predicting a compound property: for the specific processing and the technical effects of the to-be-determined compound information acquisition unit 601 and the prediction model calling unit 602, reference may be made to the relevant descriptions in the method embodiment respectively, and detailed description thereof will be omitted.

In the present embodiment exists as an apparatus embodiment corresponding to the above method embodiment. The apparatus for training a compound property prediction model and the apparatus for predicting a compound property provided in the present embodiment, by means of the first sample compound using a large sample quantity and its spatial structure information, firstly the spatial structure prediction model from which relevant knowledge of the spatial structure information is learnt is trained. Then, on the basis of the spatial structure prediction model with the relevant knowledge of the spatial structure information, the second sample compounds labeled with pieces of corresponding property information using a smaller sample quantity is used to continue training. That is, the direct correspondence between the original spatial structure and the properties is split into two parts for sequential training, making full use of a large amount of sample compound data that is not labeled with corresponding property information, so that when the number of sample compounds labeled with pieces of corresponding property information is small, a compound property prediction model having high prediction accuracy is obtained.

According to an embodiment of the present disclosure, the present disclosure also provides an electronic device, the electronic device includes: at least one processor; and a memory, communicatively connected to the at least one processor, where, the memory, storing instructions executable by the at least one processor, the instructions, when executed by the at least one processor, cause the at least one processor to implement the method for training a compound property prediction model and/or the method for predicting a compound property described in any one of the foregoing embodiments.

According to an embodiment of the present disclosure, the present disclosure also provides a readable storage medium, the readable storage medium stores computer instructions, and the computer instructions, are used to cause the computer to implement the method for training a compound property prediction model and/or the method for predicting a compound property described in any one of the foregoing embodiments.

An embodiment of the present disclosure provides a computer program product, the computer program product, when executed by a processor, can implement the method for training a compound property prediction model and/or the method for predicting a compound property described in any one of the foregoing embodiments.

FIG. 7 shows a schematic block diagram of an example electronic device 700 that can be used to implement embodiments of the present disclosure. The electronic device is intended to represent various forms of digital computers such as laptop computers, desktop computers, workstations, personal digital assistants, servers, blade servers, mainframe computers, and other appropriate computers. The electronic device may also represent various forms of mobile apparatuses such as personal digital processing, cellular telephones, smart phones, wearable devices and other similar computing apparatuses. The components shown herein, their connections and relationships, and their functions are only as examples, and not intended to limit the implementations of the present disclosure as described and/or claimed herein.

As shown in FIG. 7, the device 700 may include a computing unit 701, which may execute various appropriate actions and processes in accordance with a program stored in a read-only memory (ROM) 702 or a program loaded into a random access memory (RAM) 703 from a storage apparatus 708. The RAM 703 also stores various programs and data required by operations of the device 700. The computing unit 701, the ROM 702 and the RAM 703 are connected to each other through a bus 704. An input/output (I/O) interface 705 is also connected to the bus 704.

Multiple components in the device 700 are connected to the I/O interface 705, including: an input unit 706 including a touch screen, a touchpad, a keyboard, a mouse and the like; an output unit 707, such as various types of displays, a speaker, and the like; a storage unit 708 including a magnetic tap, a hard disk and the like; and a communication unit 709. The communication unit 709 may allow the electronic device 700 to perform wireless or wired communication with other devices to exchange data.

The computing unit 701 may be various general-purpose and/or dedicated processing components having processing and computing capabilities. Some examples of the computing unit 701 include, but are not limited to, central processing unit (CPU), graphics processing unit (GPU), various dedicated artificial intelligence (AI) computing chips, various computing units running machine learning model algorithms, digital signal processor (DSP), and any appropriate processors, controllers, microcontrollers, etc. The computing unit 701 performs the various methods and processes described above, such as the method for training a compound property prediction model or the method for predicting a compound property. For example, in some embodiments, the method for training a compound property prediction model or the method for predicting a compound property may be implemented as a computer software program, which is tangibly included in a machine readable medium, such as the storage unit 708. In some embodiments, part or all of the computer program may be loaded and/or installed on the device 700 via the ROM 702 and/or the communication unit 709. When the computer program is loaded into the RAM 703 and executed by the computing unit 701, one or more steps of the method for training a compound property prediction model or the method for predicting a compound property described above may be performed. Alternatively, in other embodiments, the computing unit 701 may be configured to perform the method for training a compound property prediction model or the method for predicting a compound property by any other appropriate means (for example, by means of firmware).

Various embodiments of the systems and technologies described in this article may be implemented in digital electronic circuit systems, integrated circuit systems, field programmable gate arrays (FPGA), application specific integrated circuits (ASIC), application-specific standard products (ASSP), system-on-chip (SOC), complex programmable logic device (CPLD), computer hardware, firmware, software, and/or their combinations. These various embodiments may include: being implemented in one or more computer programs, the one or more computer programs may be executed and/or interpreted on a programmable system including at least one programmable processor, the programmable processor may be a dedicated or general-purpose programmable processor that may receive data and instructions from a storage system, at least one input apparatus, and at least one output apparatus, and transmit the data and instructions to the storage system, the at least one input apparatus, and the at least one output apparatus.

Program codes for implementing the method of the present disclosure may be written in any combination of one or more programming languages. These program codes may be provided to a processor or controller of a general purpose computer, special purpose computer or other programmable data processing apparatus such that the program codes, when executed by the processor or controller, enables the functions/operations specified in the flowcharts and/or block diagrams being implemented. The program codes may execute entirely on the machine, partly on the machine, as a stand-alone software package partly on the machine and partly on the remote machine, or entirely on the remote machine or server.

In the context of the present disclosure, the machine readable medium may be a tangible medium that may contain or store programs for use by or in connection with an instruction execution system, apparatus, or device. The machine readable medium may be a machine readable signal medium or a machine readable storage medium. The machine readable medium may include, but is not limited to, an electronic, magnetic, optical, electromagnetic, infrared, or semiconductor system, apparatus, or device, or any suitable combination of the foregoing. More specific examples of the machine readable storage medium may include an electrical connection based on one or more wires, portable computer disk, hard disk, random access memory (RAM), read only memory (ROM), erasable programmable read only memory (EPROM or flash memory), optical fiber, portable compact disk read only memory (CD-ROM), optical storage device, magnetic storage device, or any suitable combination of the foregoing.

In order to provide interaction with a user, the systems and technologies described herein may be implemented on a computer, the computer has: a display apparatus (e.g., CRT (cathode ray tube) or LCD (liquid crystal display) monitor for displaying information to the user; and a keyboard and a pointing apparatus (for example, a mouse or trackball), the user may use the keyboard and the pointing apparatus to provide input to the computer. Other kinds of apparatuses may also be used to provide interaction with the user; for example, the feedback provided to the user may be any form of sensory feedback (for example, visual feedback, auditory feedback, or tactile feedback); and may use any form (including acoustic input, voice input, or tactile input) to receive input from the user.

The systems and technologies described herein may be implemented in a computing system (e.g., as a data server) that includes back-end components, or a computing system (e.g., an application server) that includes middleware components, or a computing system (for example, a user computer with a graphical user interface or a web browser, through which the user may interact with the embodiments of the systems and technologies described herein) that includes front-end components, or a computing system that includes any combination of such back-end components, middleware components, or front-end components. The components of the system may be interconnected by any form or medium of digital data communication (e.g., a communication network). Examples of the communication network include: local area network (LAN), wide area network (WAN), and Internet.

The computer system may include a client and a server. The client and the server are generally far from each other and usually interact through a communication network. The client and server relationship is generated by computer programs operating on the corresponding computer and having client-server relationship with each other. The server can be a cloud server, a server for a distributed system, or a server combined with blockchain.

In the technical solution of the embodiments of the present disclosure, by means of the first sample compound using a large sample size and its spatial structure information, firstly the spatial structure prediction model from which relevant knowledge of the spatial structure information is learnt is trained. Then, on the basis of the spatial structure prediction model with the relevant knowledge of the spatial structure information, the second sample compounds labeled with the pieces of corresponding property information with a smaller sample quantity is used to continue training. That is, the direct correspondence between the original spatial structure and the properties is split into two parts for sequential training, making full use of a large amount of sample compound data that is not labeled with corresponding property information, so that when the number of sample compounds labeled with pieces of corresponding property information is small, a compound property prediction model having high prediction accuracy is obtained.

It should be understood that various forms of processes shown above may be used to reorder, add, or delete steps. For example, the steps described in the present disclosure may be performed in parallel, sequentially, or in different orders, as long as the desired results of the technical solution disclosed in embodiments of the present disclosure can be achieved, no limitation is made herein.

The above specific embodiments do not constitute a limitation on the protection scope of the present disclosure. Those skilled in the art should understand that various modifications, combinations, sub-combinations and substitutions can be made according to design requirements and other factors. Any modification, equivalent replacement and improvement made within the spirit and principle of the present disclosure shall be included in the protection scope of the present disclosure.

What is claimed is:

1. A method for training a compound property prediction model, the method comprising:
for each first sample compound of first sample compounds, acquiring spatial structure information of a spatial structure formed by atoms and chemical bonds that constitute the first sample compound;
training, using the first sample compounds as input samples and pieces of corresponding spatial structure information as output samples, to obtain a spatial structure prediction model; and
continuing training, using second sample compounds as input samples and pieces of corresponding property information as output samples, to obtain the compound property prediction model on a basis of the spatial structure prediction model, wherein an order of magnitudes of the second sample compounds labeled with the pieces of corresponding property information being less than an order of magnitudes of the first sample compounds that are not labeled with corresponding property information.

2. The method according to claim 1, wherein acquiring spatial structure information of the spatial structure formed by atoms and chemical bonds that constitute the first sample compound, comprises:
acquiring the atoms and the chemical bonds, formed by the atoms, constituting the first sample compound;
through a molecular dynamics simulation or a experimental calculation, determining three-dimensional coordinates of respective atoms, bond angles between different chemical bonds, atomic distances between the atoms, and an overall potential energy presented by the atoms and the chemical bonds; and
using at least one of the three-dimensional coordinates, the bond angles, the atomic distances, and the overall potential energy as the spatial structure information of the first sample compound.

3. The method according to claim 1, wherein the property information of a compound comprises at least one of water solubility, toxicity, a matching degree with preset protein, compound reaction characteristics, stability, or degradability.

4. The method according to claim 1, wherein continuing training, using second sample compounds as input samples and pieces of corresponding property information as output samples, to obtain the compound property prediction model on the basis of the spatial structure prediction model, comprises:
controlling, in a fine-tune manner, the spatial structure prediction model to learn a correspondence from a sample pair of a second sample compound used as an input sample and a piece of corresponding property information used an the output sample, to obtain the compound property prediction model.

5. The method according to claim 1, further comprising:
distillating, in response to a complexity of the spatial structure prediction model exceeding a preset complexity, to obtain a lightweight spatial structure prediction model through a model distillation technology.

6. The method according to claim 1, further comprising:
acquiring a to-be-determined compound with properties to be determined; and
calling the compound property prediction model to predict property information of the to-be-determined compound.

7. An apparatus for training a compound property prediction model, the apparatus comprising:
at least one processor; and
a memory storing instructions, wherein the instructions when executed by the at least one processor, cause the at least one processor to perform operations, the operations comprising:
for each first sample compound of first sample compounds, acquiring spatial structure information of a spatial structure formed by atoms and chemical bonds that constitute the first sample compound;
training, using the first sample compounds as input samples and pieces of corresponding spatial structure information as output samples, to obtain a spatial structure prediction model; and
continuing training, using second sample compounds as input samples and pieces of corresponding property information as output samples, to obtain the compound property prediction model on a basis of the spatial structure prediction model, wherein an order of magnitudes of the second sample compounds labeled with the pieces of corresponding property information being less than an order of magnitudes of the first sample compounds that are not labeled with corresponding property information.

8. The apparatus according to claim 7, wherein the operations further comprise:
acquiring the atoms and the chemical bonds, formed by the atoms, constituting the first sample compound;
through a molecular dynamics simulation or a experimental calculation, determining three-dimensional coordinates of respective atoms, bond angles between different chemical bonds, atomic distances between the atoms, and an overall potential energy presented by the atoms and the chemical bonds; and
using at least one of the three-dimensional coordinates, the bond angles, the atomic distances, and the overall potential energy as the spatial structure information of the first sample compound.

9. The apparatus according to claim 7, wherein the property information of a compound comprises at least one of water solubility, toxicity, a matching degree with preset protein, compound reaction characteristics, stability, or degradability.

10. The apparatus according to claim 7, wherein the operations further comprise:
controlling, in a fine-tune manner, the spatial structure prediction model to learn a correspondence from a sample pair of a second sample compound used as an input sample and a piece of corresponding property information used as an output sample, to obtain the compound property prediction model.

11. The apparatus according to claim 7, the operations further comprising:
distillating, in response to a complexity of the spatial structure prediction model exceeding a preset complexity, to obtain a lightweight spatial structure prediction model through a model distillation technology.

12. The apparatus according to claim 7, the operations comprising:
acquiring a to-be-determined compound with properties to be determined; and
calling the compound property prediction model to predict property information of the to-be-determined compound.

13. A non-transitory computer readable storage medium, storing computer instructions, the computer instructions, being used to cause the computer to perform operations comprising:
for each first sample compound of first sample compounds, acquiring spatial structure information of a spatial structure formed by atoms and chemical bonds that constitute the first sample compound;
training, using the first sample compounds as input samples and pieces of corresponding spatial structure information as output samples, to obtain a spatial structure prediction model; and
continuing training, using second sample compounds as input samples and pieces of corresponding property information as output samples, to obtain a compound property prediction model on a basis of the spatial structure prediction model, wherein an order of magnitudes of the second sample compounds labeled with the pieces of corresponding property information being less than an order of magnitudes of the first sample compounds that are not labeled with corresponding property information.

14. The non-transitory computer readable storage medium according to claim 13, the operations further comprising:
acquiring the atoms and the chemical bonds, formed by the atoms, constituting the first sample compound;
through a molecular dynamics simulation or a experimental calculation, determining three-dimensional coordinates of respective atoms, bond angles between different chemical bonds, atomic distances between the atoms, and an overall potential energy presented by the atoms and the chemical bonds; and
using at least one of the three-dimensional coordinates, the bond angles, the atomic distances, and the overall potential energy as the spatial structure information of the first sample compound.

15. The non-transitory computer readable storage medium according to claim 13, wherein the property information of a compound comprises at least one of water solubility, toxicity, a matching degree with preset protein, compound reaction characteristics, stability, or degradability.

16. The non-transitory computer readable storage medium according to claim 13, the operations further comprising:
controlling, in a fine-tune manner, the spatial structure prediction model to learn a correspondence from a sample pair of a second sample compound used as an input sample and a piece of corresponding property information used as an output sample, to obtain the compound property prediction model.

17. The non-transitory computer readable storage medium according to claim 13, the operations further comprising:
distillating, in response to a complexity of the spatial structure prediction model exceeding a preset complexity, to obtain a lightweight spatial structure prediction model through a model distillation technology.

18. The non-transitory computer readable storage medium according to claim 13, the operations further comprising:
acquiring a to-be-determined compound with properties to be determined; and
calling the compound property prediction model to predict property information of the to-be-determined compound.
